(19) United States
(12) Patent Application Publication
(10) Pub. No.: US 2010/0330601 A1
(43) Pub. Date: Dec. 30, 2010

(54) CONTROL SYSTEM FOR UV LAMPS, AND CHECK SYSTEM FOR DETERMINING THE VIABILITY OF MICROORGANISMS

(76) Inventors: Antonius Marinus Telgenhof Oude Koehorst, Hengelo (NL); Petrus Gerhardus Maria Wolberink, Groenlo (NL); Jacobus Cornelis Musters, Swifterbant (NL); Franciscus Peter Houwen, Kronenberg (NL)

Correspondence Address: RISSMAN HENDRICKS & OLIVERIO, LLP, 100 Cambridge Street, Suite 2101, BOSTON, MA, 02114, US

(21) Appl. No.: 12/377,486

(22) PCT Filed: Aug. 13, 2007
(86) PCT No.: PCT/NL07/50401

§ 371 Date: Aug. 26, 2010

(30) Foreign Application Priority Data
Aug. 14, 2006 (NL) 1032315

(51) Int. Cl. C12Q 1/02; C12M 1/36

(52) U.S. Cl. 435/29; 435/286.1

(57) ABSTRACT

The invention relates to a control system for a method for controlling at least one UV lamp for treating a liquid such as water, wherein a biosensor is used. In addition, the invention relates to the use of biosensors for detecting or monitoring viable cells. The invention uses one or more viability parameters.

The present invention relates to a check system, comprising a measuring and/or control system, for UV lamps for treating liquids, in particular water and more specifically drinking water, and to a check system with which the viability of microorganisms and in particular bacteria can be monitored.

The treatment of wastewater and drinking water with ultraviolet light is developing as a powerful means for inactivating microorganisms. An example of such liquid treatment systems is given by American patent application US-A1-2004/0118786. In this patent application, also a number of other publications are mentioned which also relate to apparatuses and systems for using UV lighting in order to inactivate microorganisms.

In such water purification systems, UV lamps are placed in, above or around a reactor, while water flows through the reactor and is lighted therein. As a rule, the UV lamps are perpendicular to the flow direction of the water. Incidentally, the reactor may then be an open channel.

This process is generally carried out continuously, while the liquid to be treated remains in the lighting area for some time (this time is also referred to as residence time or also retention time). Unlike, for instance, by adding a microorganism-inactivating compound, such as chlorine, a lighting by UV has no residual effect, in the sense that, when the lamp is not active anymore, the inactivation does not occur either.

It is not so much that the microorganisms are eliminated by the lighting with ultraviolet radiation. The prevention of reproduction of microorganisms is sufficient. When this specification and the appended claims refer to “viability” of particular microorganisms, what is intended is that the respective microorganisms are (still) capable of reproducing and thereby developing into a population which is capable of bringing about adverse effects. For measuring the viability, in this specification, in fact the extent is determined to which a microorganism produces a signal during measurement of a particular microbiological or biochemical characteristic.

The lighting with UV lamps is relatively expensive. The lamps require a relatively high energy consumption and only have a limited life and/or a limited number of burning hours.

Therefore there is a wish to save costs on power consumption and on the life of UV lamps. However, this is only possible if a quick check or control system is available, with which the effect of the lighting on the viability of the microorganisms can be guaranteed.

In addition, in case of failure of the UV lamps and with the absence of residual effect of the treatment with UV beams, the system is more vulnerable than when, for instance, chlorine is used. A quick check system, in particular a quick measuring and control system with effective checkpoints or measuring points is then required as well.

It is a primary object of the present invention to provide such a check system and in particular such a measuring and control system. In other words, the invention contemplates a control system which couples applied doses of ultraviolet light to a sufficient degree of inactivation of microorganisms.

The guidelines for maintaining the bacteriological quality of water, and more specifically drinking water, are based on bacterial growth. Here, so-called indicator organisms are taken as being indicative. In practice, checks for viability are (still) carried out by taking samples of the treated liquid and plating these out on a suitable nutrient medium in, for instance, a Petri dish. This means that an inoculate is spread over a solid substrate, such as an agar gel, and is thereby diluted such that microorganisms present are individualized, after which each individual microorganism, such as a bacterium, can develop into a colony, which is visible to the naked eye. In the solid nutrient medium, nutrients, salts, etc. are added, which enable the development of particular organisms. If one or more colonies are formed, viable microorganisms are present. Incidentally, sometimes viable organisms are present, while still no growth occurs within 48 hours. These organisms are then, for instance, in a state of dormancy.

However, bacterial growth is a process which is (too) slow. As a rule, the development from a single bacterium to a colony visible to the naked eye takes about 18-48 hours.

In order to automate all this, for instance, in European patent application EP-A-0 682 244, after a description of the above problems, it is disclosed to monitor the viability process of particular indicator organisms by color measurements, for instance on the basis of enzymes.

The present invention proposes to use a biological sensor, with which the viability of bacteria can be determined. With such a sensor, for instance, the regulation of the UV lamps can be controlled. However, such a sensor needs to yield results, and consequently produce a signal, within a short period of time of less than three hours, and preferably less than two hours, for instance within one hour after sampling.

This sensor is not based on biological growth. The present invention is directed to viability parameters, namely microbiological and/or biochemical characteristics which are a measure of the viability of bacteria. En this light, it is noted that there is not one correlation which is clear in all circumstances between the measured viability and the degree to which a bacterial population is still capable of reproducing. For instance, (after a particular treatment) the correlation between a value of a viability parameter and the extent of growth will change with changing conditions. Possibilities to be considered here are a growth medium, growth temperature, but also the previous history of the bacteria. For instance, drinking water is a poor environment, while meat extract is a very rich environment. Further, bacteria may be in a state of dormancy due to different conditions. Therefore, viability parameters will not always be an absolute measure of the viability/vitality/activity of the indicator bacterium. In other words, viability parameters are a measure of the actual viability, at least the viability parameters are correlated to the actual viability, or growth potential; and on the basis of a viability parameter, a prediction can be made for the actual viability.

In microbiology, inter alia the following viability parameters are used: the integrity of the membrane of the microorganism, the membrane potential, the respiration, and the enzyme activity. However, these four viability parameters should not be taken as being limiting for the present invention in any way. Of the last parameter, the technique described in EP-A-0 682 244 is an example.

Further, in U.S. Pat. No. 5,821,066, use is made of the respiration of microorganisms. In particular, this patent relates to a quick method for detecting, identifying and counting respiring microorganisms, by contacting these microorganisms either with a fluorochromic dye in combination with fluorescent antibodies or with immunomagnetic beads and quantifying respiring microbial cells after incubation.

The present inventors have found that the effect of (particular the power of) the ultraviolet irradiation on the viability of the microbial cells strongly influences the usability of the method. In other words, the inventors have found a method where determining one or more viability parameters is sufficiently indicative for regulating UV lamps. In particular, a sensitization of the microorganisms is required. The effect of ultraviolet irradiation on viability parameters is influenced, so that the determination of the respective viability parameter(s) is usable in a check system and particularly in a measuring or control system.

In a first aspect, the invention therefore relates to a control system for at least one UV lamp for treating a liquid, in particular water and more specifically drinking water, comprising, in addition to the at least one UV lamp, means for concentrating microorganisms from a sample of that liquid; means for sensitizing the microorganisms; measuring means for determining at least one viability parameter; and control means for switching on or switching off the at least one UV lamp, or controlling the power of that at least one UV lamp, on the basis of the viability parameter determination.

In a second aspect, the invention relates to a method for controlling at least one UV lamp for treating a liquid, in particular water and more specifically drinking water, comprising taking a sample of this liquid; concentrating the microorganisms from that sample; sensitizing the microorganisms; determining at least one viability parameter; and switching on or switching off the at least one UV lamp, or controlling the power thereof, on the basis of the viability parameter determination.

Both in the system and in the method according to the invention, first a sample needs to be taken from the treated liquid, from which the microorganisms, and particularly the bacteria present therein, are concentrated. This concentration can suitably be carried out by carrying out a filtration, with the microorganisms remaining on the filter. Very suitably, use can then be made of a ceramic microfiltration membrane, but other bacteria filters may be used as well.

As already noted hereinabove, viability parameters are not always an absolute measure of the viability, vitality or activity of microorganisms. However, by making the viability determination relative with respect to a second determination, it will still be sufficiently informative: the extent to which the viability changes says enough about the reproduction potential of the bacterial population(s).

This making relative may, for instance, be done by measuring:
before and after a treatment
at multiple times in the same place
in one place directly after a treatment and at some distance therefrom.

For many embodiments, it is therefore advisable to take a sample both before and after the treatment with UV, so that the effect of the treatment can be checked, i.e. measured, therewith.

After the concentration step, the collected microorganisms may optionally be washed.

Before discussing the sensitization, reference is made to the study incorporated hereinbelow.

The inventors used a study where the bacterium Escherichia coli (hereinafter: E. coli), a very conventional indicator organism for water quality, was irradiated with different doses of ultraviolet radiation. Here, attention was paid to growth and the four above-mentioned viability parameters, namely membrane integrity, membrane potential, respiration and enzyme activity.

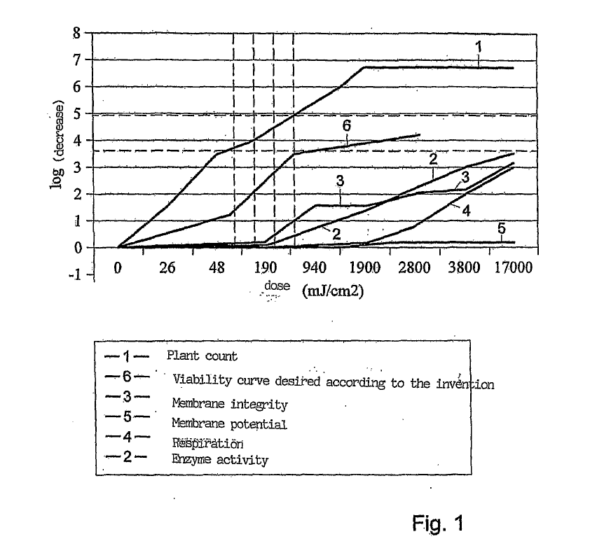

FIG. 1 graphically shows the results of that study. In FIG. 1, the logarithm of the decrease in growth, or viability parameter is plotted against the UV dose in mJ/cm². More in detail, curve 1 indicates the degree of elimination of E. coli determined with the classic plate method, with the flat part of the curve approaching 100% elimination. Curve 2 shows the decrease of the signal corresponding with the viability parameter enzyme activity; curves 3-5 show the change or the dependence of the signal of the viability parameters membrane integrity, respiration and membrane potential, respectively. These viability parameters are determined in a known manner by means of specific color reactions which result in detectable fluorescence of the bacteria. After detection, the digital image obtained is analyzed by means of software. Curve 6 shows the viability curve, desired according to the invention.

More in detail, the curves 2-5 in FIG. 1 show the different sensitivities of the different viability parameters to ultraviolet light. The range of the UV doses whereby 99.97% and more microorganisms are damaged in such a manner that they can no longer grow on a plate (see curve 1, from log value 3.5), does not coincide with the range of the doses whereby the different viability parameters are influenced. Further, the curves 2-5 show a large mutual difference in sensitivity of the parameters studied to UV light.

The present inventors have realized, and the present invention is directed to this realization, that, in practice, the control system needs to be sensitive with treatment with UV light with a dose in the range of about 60 mJ/cm² to about 600 mJ/cm². In that range, a degree of elimination or deactivation needs to take place with a factor of about 10³-10⁵. This requires that the sensitivity of the viability parameter needs to be adjusted, such that the curve shifts to the indicated desired curve 6. Here, it should be noted that curve 6 is only an exemplary form. In the respective range of UV doses, the curve needs to be sufficiently steep and be preferably linear.

This adjustment of the sensitivity of the viability parameter now Forms the essence of the present invention, and is referred to as “sensitization”. This sensitization occurs by contacting the microorganisms with particular (chemical) compounds, such as molecules or compounds with a (bio)chemical effect and/or by treating them with physical techniques, with the purpose of positively or negatively influencing the determination of one or more viability parameters of microorganisms. Examples of physical techniques are subjecting the microorganisms to a temperature shock such is a heat or cold shock, subjecting them to a (strong) magnetic and/or electric field, for instance a magnetic shock or current surge is applied. Examples of a treatment with chemical compounds comprise applying a pH shock, using different salt concentrations, or adding a molecule or, in general, a chemical compound which (directly) has a specific effect on the determination of a viability parameter, such as compounds making cell membrane permeable, with isopropanol as an example.

Incidentally, not every microorganism needs to be tested. When checking, for instance, water, it is accepted to monitor one or more indicator organisms, such as E. coli. Here, it goes without saying that it is necessary then to identify that indicator organism, for instance E. coli, as such with, for instance, fluorescent antibodies.

The viability tests are carried out in a manner known per se, for instance by means of specific color reactions which result in detectable fluorescence of the bacteria. After detection, the digital image obtained is analyzed by means of software. Depending on the signal, a UV lamp may or may not be switched on or off or the power of the lamp may be adjusted. Here, a skilled person will be able to simply determine the threshold values needed for his specific system.

A protocol which has brought the inventors to their invention consists of a carrousel with four “determination locations”. At a location, what is successively done is:
collecting, for instance, 100 ml of sample, for instance a water sample;
filtering the sample through a special filter which stops the indicator organisms, while there is still sufficient flow. An example of such a filter has a diameter of, for instance, 8 cm; pore size 0.2 μm-0.4 μm; filtering time 10 min-30 min;
washing the filter with the indicator organisms thereon one or more times with a buffer solution.

From this moment, it is preferred to keep the system at a constant temperature (in the range of 20° C.-37° C.).
optionally, the indicator organisms may be incubated in this buffer solution for, for instance, 0 min-30 min;
adding dye(s); these may, for instance, be added (in dissolved formed) to the buffer solution already present (mixing required) or after suctioning off the buffer solution;
incubating the indicator organisms with dye(s).

According to the invention, this incubation may be preceded by a sensitization step, where, for instance, the molecules or compounds with a (bio)chemical effect are added to the buffer already present, or where this buffer solution is suctioned off first. The sensitization step may optionally also take place during the incubation with dye(s).

In addition to addition of molecules or compounds with a (bio)chemical effect, use may also be made of physical techniques. Sensitization with physical techniques may also take place before and/or during the incubation with dye(s);
identification of the indicator organisms by incubating with, for instance, a specific antibody, provided with an inducible fluorescent chemical group.

The incubation with, for instance, antibodies may, incidentally, take place before the incubation with dye(s) in the washing buffer, during the incubation with dye(s), or after the incubation with dye(s) in fresh washing buffer or in a different buffer.

After detection, the digital image obtained is analyzed by means of software. This may result in either an absolute measure or a relative measure of the viability. Depending on the signal, a UV lamp may be or may not be switched on or off or the power of the lamp may be adjusted. The regulation of the lamps will also be done by means of software, while a skilled person sets the necessary settings, for instance threshold values, in the software.

Incidentally, the sensor may also be used as a check means at (some) distance behind a UV irradiation installation. If then an increase in viability or in the number of viable indicator organisms is determined, desired measures can be taken.

As a last step of the method according to the invention, optionally the filter may be regenerated for a next sampling.

Incidentally, in another embodiment of the method according to the invention, multiple viability parameters are determined in or on the same sample, either simultaneously or successively. This can make the correlation between the viability parameters and the growth potential more indicative.

Although, in the first two aspects, the invention is coupled to controlling UV radiation, the invention is also usable in, for instance, the treatment of liquids such as water with chemicals, such as chlorine, where inactivation of microorganisms occurs as well, while the invention is also usable in the bacteriological inspection of media, such as water purifications, water purification in horticultural greenhouses, rinse water in flower bulb cultivation and vegetable cultivation, wastewater of the preservative industry, fishponds, and media used or generated in the food industry.

In addition, the invention may also extend to other cells than microorganisms and it is usable to, for instance, determine the activity of particular body cells, for instance after administering specific medicines. Further, the viability of cells and microorganisms in blood can be monitored.

The biosensor may also be used to determine whether the activity of non-suitable or desired organisms increases. Further, microorganisms added to a liquid may be monitored.

In a last aspect, the invention therefore relates to a method and system for detecting viable microorganisms, comprising detecting viable cells, such as microorganisms and body cells, comprising providing sufficient cells; sensitizing these cells, and determining at least one viability parameter. The provision of sufficient cells sometimes means, depending on the determination, a concentration step by means of, for instance, filtering, sometimes a diluting step (for instance with a determination of blood), and sometimes, for instance, a tissue specimen without concentration dilution.

The invention will now be illustrated in more detail on the basis of the following non-limiting example.

### EXAMPLE

In this example, the invention is illustrated for the sample organism E. coli, which was subjected to irradiation with UV light. As a viability parameter, the membrane integrity was chosen.

The membrane integrity was measured by offering propidium iodide, which is a relatively large molecule, externally (outside the cell), in the medium in which E. coli is present. With a healthy bacterium, propidium iodide cannot pass the intact cell membrane and will therefore only penetrate those bacteria which have a permeable cell membrane. In the bacterial cell, propidium iodide becomes attached to the DNA present, so that the fluorescent capacity of this molecule is increased by a factor 1000. So, a positive cell staining means that the cell is not viable.

The dose of UV light was varied, and the results are in the following Table.

| Dose of ultraviolet light (mJ/cm²) | Propidium iodide |
|---|---|
| 0 | virtually no fluorescence |
| 150 | virtually no fluorescence |
| 300 | virtually no fluorescence |
| 600 | very slight fluorescence |
| 750 | slight fluorescence |
| 1500 | fluorescence |

The results were assessed on the basis of microscopic images without aid of data analysis software. Staining with propidium iodide differs between irradiations with different doses of UV light, albeit from about 500 mJ/cm².

In order to make the parameter usable for the present invention, then the test was repeated, but now only after first isopropanol was added and, after washing, then propidium iodide. Isopropanol can make the bacterial cell membranes permeable to large molecules such as propidium iodide. Here, it was found that higher concentrations of isopropanol as well as a longer incubation period result in an increase of the fluorescence.

More in detail, it was found that if, after the irradiation of E. coli with different doses of ultraviolet radiation, incubation took place with a particular concentration of isopropanol (18%), the following result was obtained:
a treatment with isopropanol for 10 minutes, followed by incubation with propidium iodide resulted in increasing fluorescence: <150≈300<450≈600 mJ/cm². So, a difference in the degree of fluorescence is visible between 0 and 150 mJ/cm² and between 300 and 450 mJ/cm²;
a same treatment with isopropanol for 60 minutes yields a similar result: 0<150<300≈450≈600≈750 mJ/cm². The longer incubation with (the same concentration of) isopropanol caused an extra shift of the sensitivity, being the extent to which a signal changes as a result of a change in its cause. In other words, it is found to be possible to get the range shifted in which the change of the signal is sufficiently sensitive to UV irradiation.

It is concluded that it is possible to demonstrate a difference of the effect of different doses of ultraviolet light, in the range between 0 and 450 mJ/cm², on the viability parameter membrane integrity.

1. A control system for at least one UV lamp for treating a liquid in addition to the at least one UV lamp, means for concentrating microorganisms from a sample of that liquid; means for sensitizing the microorganisms; measuring means for determining at least one viability parameter; and control means for, on the basis of the viability parameter determination, switching on or switching off the at least one UV lamp, or regulating the power of the at least one UV lamp.

2. A control system according to claim 1, wherein the measuring means for determining at least one viability parameter are chosen from measuring means for determining enzyme activity, membrane integrity, respiration and/or membrane potential.

3. A control system according to claim 1, wherein the means for sensitizing the microorganisms are chosen from chemical agents physical processes.

4. A control system according to claim 1, wherein the measuring means provide a reading which, via a microprocessor, is converted into a control signal for the at least one UV lamp.

5. A control system according to claim 1, wherein the measuring means provide a reading which, via a microprocessor, is converted into a control signal for the at least one UV lamp.

6. A method for controlling at least one UV lamp for treating a liquid, comprising taking a sample of the liquid, concentrating the microorganisms from the sample; sensitizing the microorganisms; determining at least one viability parameter; and, on the basis of this determination switching on or off the at least one UV lamp, or regulating the power thereof.

7. A method according to claim 6, wherein the viability parameter is chosen from enzyme activity, membrane integrity, respiration and/or membrane potential.

8. A method according to claim 6, wherein the sensitization of the microorganisms is performed by adding chemical agents, or by carrying out physical processes.

9. A method according to claim 6, wherein color measurements are carried out for determining the viability parameter.

10. A method according to claim 6, wherein the measuring means provide a reading which via a microprocessor, is converted into a control signal for the at least one UV lamp.

11. A method for detecting or monitoring viable cells, comprising providing sufficient cells; sensitizing these cells, and determining at least one viability parameter.

12. A control system according to claim 1, wherein the liquid is water.

13. A control system according to claim 1, wherein the liquid is drinking water.

14. A control system according to claim 3, wherein the chemical agents are selected from molecules or compositions with a chemical or biochemical effect.

15. A control system according to claim 14, wherein the molecules or compositions with a chemical or biochemical effect are compounds that make a membrane permeable.

16. A control system according to claim 3, wherein the physical processes are selected from heat shock and cold shock generators, and magnetic and/or electric fields.

17. A method according to claim 6, wherein the liquid is water.

18. A method according to claim 6, wherein the liquid is drinking water.

19. A method according to claim 8, wherein the chemical agents are selected from molecules or compositions with a chemical or biochemical effect.

20. A method according to claim 8, wherein the physical processes are selected from heat shock and cold shock generators, and magnetic and/or electric fields.

21. A method according to claim 8, wherein the viable cells are microorganisms.
